(19) United States
(12) Patent Application Publication
(10) Pub. No.: US 2007/0059352 A1
(43) Pub. Date: Mar. 15, 2007

(54) Therapeutic liposomes

(76) Inventors: Gerard Jensen, Brea, CA (US); Ning Hu, San Gabriel, CA (US); Danny Petrasek, Los Angeles, CA (US)

Correspondence Address: VIKSNINS HARRIS & PADYS PLLP, P.O BOX 111098, St. Paul, MN, 55111-1098, US

(21) Appl. No.: 11/370,008

(22) Filed: Mar. 6, 2006

Related U.S. Application Data
(63) Continuation of application No. PCT/US04/29265, filed on Sep. 9, 2004.
(60) Provisional application No. 60/501,818, filed on Sep. 9, 2003.
(60) Provisional application No. 60/501,816, filed on Sep. 9, 2003.

(51) Int. Cl. A61K 9/127; A61K 31/70; A61K 33/24

(52) U.S. Cl. 424/450; 424/617; 514/23

(57) ABSTRACT

The invention provides methods for correcting an imbalance of one or more entities in a mammal, as well as therapeutic liposomes and compositions for use with such methods.

### CROSS-REFERENCE TO RELATED APPLICATIONS

This application is a continuation under 35 USC 111(a) of PCT/US2004/029265 filed Sep. 9, 2004 and published in English on Jun. 16, 2005 as WO 2005/053605 A2, which claimed priority from U.S. Provisional Application Nos. 60/501,818 and 60/501,816 both filed Sep. 9, 2003, which applications and publications are incorporated herein by reference.

### FIELD OF THE INVENTION

The present invention relates to liposomes that are capable of correcting an imbalance of one or more entities in an animal.

### BACKGROUND OF THE INVENTION

Numerous diseases and conditions are caused by or lead to an imbalance of one or more entities in an animal. Hypercalcemia is a common clinical metabolic problem. It can occur as a manifestation of many disorders including: hyperparathyroidism, pagets disease, vitamin A and vitamin D toxicity, tuberculosis, sarcoidosis, malignancies e.g. breast cancer, small cell lung cancer, squamous cell carcinoma, multiple myeloma, and others.

Hypercalcemia occurs in 10%-20% of people with cancer, making it one of the most common medical management problems facing physicians. Moreover, hypercalcemia is the most common life-threatening metabolic disorder associated with malignancy. Although there are a variety of clinical options for treating hypercalcemia, many are limited in application and produce undesired side effects that make treatment difficult and unpleasant for the patient. Accordingly, there is a need for additional methods for treating hypercalcemia.

Diabetes is a condition that is characterized by an absolute or relative deficiency of insulin, which leads in more severe cases, to chronic hyperglycemia. The long term complications of diabetes include development of neuropathy, retinopathy, nephropathy, and an increased susceptibility to infection (Stedman's Medical Dictionary, 26 ed., Williamw & Wilkins, Baltimore, Md., 1995). There is currently a need for additional methods for treating hyperglycemia, including hyperglycemia that results from diabetes. This is especially the case for hyperglycemia that does not respond to insulin containing regimens.

An excess amount of metal ions is also associated with a variety of diseases and conditions. For example, excess amounts of manganese, iron, mercury, aluminum, and copper have all been associated with Parkinson's disease. Parkinson's disease involves the deterioration of specific nerve centers in the brain. It is believed that these excess minerals increase free radical pathology and accelerate cell death. This deterioration changes the chemical balance of two neurotransmitters essential for transmission of nerve signals. The ultimate result is lack of control of physical movements.

Another disease related to high levels of metals in the blood is Alzheimer's disease. Alzheimer's disease is a progressive condition that destroys brain cells and structures. Researchers believe the disease is associated with excess zinc, copper, aluminum, and/or iron in the blood and/or brain. People with Alzheimer's disease slowly lose their ability to learn, remember and function.

Another disease associated with excess metal ions in the body is Wilson's disease. Wilson's disease is caused by a build up of excess copper in the liver, brain, and kidneys. The disease is characterized by inflammation and cirrhosis of the liver and brain damage. If untreated, Wilson's disease is fatal. The accumulation of copper in the brain has also been associated with various forms of Transmissible Spongiform Encephalopathy (TSE) including Creutzfeldt-Jakob Disease (CJD).

In addition, exposure to heavy metals has been linked to developmental retardation, various cancers, kidney damage. For example, exposure to mercury, gold, and lead has been associated with the development of autoimmunity, a condition in which the immune system attacks its own cells, mistaking them for foreign invaders. Exposure to heavy metals such as mercury, lead, cadmium, and aluminum is also believed to be associated with increased free radical damage to the central nervous system and multiple sclerosis.

There is currently a need for methods that are useful for removing unwanted or deleterious entities from animal systems and from biological samples.

### SUMMARY OF THE INVENTION

The present invention provides liposomes that are capable of correcting an imbalance of one or more entities in an animal. Accordingly, the invention provides a method for incorporating an entity from an animal into a liposome comprising administering to an animal in need of such treatment liposomes capable of incorporating the entity.

The invention also provides a method for incorporating an entity from a biological sample into a liposome comprising contacting the biological sample with one or more liposomes capable of incorporating the entity.

The invention also provides a method for removing an entity from the cerebral spinal fluid of an animal in need of such treatment comprising interthecally administering to the animal liposomes capable of incorporating the entity.

The invention also provides a method for reducing serum calcium load in an animal in need of such treatment comprising administering an effective calcium reducing amount of one or more liposomes.

The invention also provides a method for treating Alzheimer's disease in an animal comprising administering an amount of a liposome to the animal that is effective to lower the level of Zn, Al, Fe or Cu, or an ion thereof in the animal.

The invention also provides a method for treating Parkinson's disease in an animal comprising administering an amount of a liposome to the animal that is effective to lower the level of Mn, Fe, Hg, Al, or Cu, or an ion thereof in the animal.

The invention also provides a method for treating hyperglycemia in an animal comprising administering an amount of a liposome to the animal that is effective to lower the level of glucose in the animal.

The invention also provides a liposome as described herein for use in medical therapy.

The invention also provides the use of a liposome as described herein to prepare a medicament useful for reducing serum calcium load in an animal.

The invention also provides the use of a liposome as described herein to prepare a medicament useful for treating Alzheimer's disease in an animal.

The invention also provides the use of a liposome as described herein to prepare a medicament useful for treating Parkinson's disease in an animal.

The invention also provides the use of a liposome as described herein to prepare a medicament useful for treating diabetes in an animal.

### BRIEF DESCRIPTION OF THE FIGURES

FIG. 1, illustrates the lipid transition temperature dependence of calcium loading for various preparations.

FIG. 2 illustrates calcium loading for three formulations prepared using different methods.

FIG. 3 illustrates the influence of pH and NaCl on calcium loading efficiency.

FIG. 4 illustrates calcium loading for HSPC:cholesterol formulations.

FIG. 5 illustrates loading efficiencies for HSPC:cholesterol formulations.

FIG. 6 illustrates calcium loading for DOPC:cholesterol formulations.

FIG. 7 illustrates loading efficiencies for DOPC:cholesterol formulations.

FIG. 8 illustrates calcium loading for DEPC:cholesterol formulations.

FIG. 9 illustrates loading efficiencies for DEPC:cholesterol formulations.

FIG. 10 illustrates calcium loading for DPPC:cholesterol formulations.

FIG. 11 illustrates loading efficiencies for DPPC:cholesterol formulations.

### DETAILED DESCRIPTION OF THE INVENTION

Definitions

As used herein, the term “sequestering agent” or “receiver” refers to compounds complexes, molecules, or atoms capable of binding to an entity thereby removing the entity from the immediate surroundings.

As used herein, the term “animal” refers to mammals, birds, reptiles, and fishes.

As used herein the term “biological sample” includes a tissue, serum, blood, plasma, cerebral spinal fluid, saliva, urine, etc. sample taken from an animal.

Entities

The invention provides methods for removing unwanted entities from animals or from biological samples. Liposomes can be formulated to incorporate a wide variety of entities. Accordingly, the methods of the invention are generally useful for removing a wide variety of materials from an animal or from a biological sample. For example, the methods of the invention can be used to remove metal or a metal ions e.g. an alkali metal, an alkaline earth metal, Fe, Os, Co, Ni, Pd, Cu, Ag, Au, Zn, Al, Cd, Hg, Sn, or Pb, or an ion thereof). In particular, the methods of the invention are useful for removing Zn, Al, Fe or Cu, or an ion thereof from an animal. In a preferred embodiment, the methods are useful for removing calcium from an animal.

The methods are also useful for removing unwanted molecules (e.g. peptides, organic molecules, therapeutic agents, nitrous oxide, or glucose) from an animal. Accordingly, the methods are useful for therapy (i.e. removing peptides or compounds that are associated with a pathological condition). The methods are also useful for treating over exposure to therapeutic agents (i.e. overdoses) or toxic substances (i.e. poisonings). As used herein, the term organic compound includes any compound that comprises one or more carbon atoms. Typically an organic compound has a molecular weight of less than about 450 atomic mass unit (amu). In one preferred embodiment, the organic compound has a molecular weight of less than about 300 amu.

Contact with the Liposome

The methods of the invention can be carried out in vitro or in vivo. For example, the methods can typically be carried out by administering liposomes to an animal. In some situations, it may be more convenient to remove a biological sample (e.g. serum, cerebral spinal fluid, or tissue) from the animal and contact the sample with the liposomes outside the animal. Following removal of the undesirable entity, the sample can be returned into the animal. The methods of the invention are useful for therapeutic applications as well as for diagnostic applications.

Incorporation into the Liposome

When used to incorporate a metal or a metal ion, the liposomes used in the methods of the invention typically include an ion channel or shuttle to facilitate entry of the metal or the ion into the liposome. For example, A23187 (available from CalBiochem (La Jolla, Calif.) or from Fermentek Ltd (Jerusalem, Israel) can be used to transport Ca⁺² or other divalent cations. Such a channel or shuttle, however, is not always required. For example, amphiphilic entities can load in response to a chemical potential gradient (e.g. against a pH gradient). Additionally, when used to incorporate hydrophobic entities, a channel that facilitates entry of the hydrophobic entity may or may not be necessary. The liposome may be permeable to some hydrophobic entities, but the entity may be trapped or modified inside the liposome after entry, so that it does not immediately pass back out of the liposome.

Once incorporated into the liposome, the entity may simply reside in the hydrophilic environment of the liposome interior, in the hydrophobic bilayer, or the liposome may include a sequestering agent that associates with (e.g. through hydrogen bonding or ionic interactions) the entity and reduces its ability to pass out of the liposome. For example, the liposome may include sequestering agents such as a polyamine, polydentate carboxylic acid (e.g. EDTA), crown ether, lactam, an inorganic compound, or other agents that create chemical gradients to draw the entity into liposomes. The liposomes may also include a reagent or enzyme capable of reacting with the entity so as to reduce its ability to pass out of the liposome. For example glucose could undergo conversion within the liposome to glucose-6-phosphate using hexokinase. If the reaction in the liposome requires an energy source (e.g. ATP), a co-factor, a specific pH, or a specific ion balance to take place, the liposomes can be prepared so that they includes these features. For in vitro applications, such features may also be provided from an external source.

Mechanism of Liposome Action

According to the methods of the invention, the liposomes can reduce the available concentration of an entity in an animal in a number of ways. For example, 1) the liposome can incorporate the entity in a reversible manner and then release the entity over time so that the maximum amount or concentration of entity available in the animal over that time is reduced; 2) the liposome can incorporate the entity and then be cleared from the animal thereby eliminating the entity from the animal's serum; 3) the liposome can incorporate the entity and the entity can be sequestered or modified within the liposome so that it does not pass out of the liposome; or 4) following incorporation of the entity, the liposome can be redirected to other locations in the body (e.g. loaded into a macrophage) where the entity can be released or where the liposome containing the entity can be eliminated from the body. A combination of one or more of 1-4 above may also occur.

Combination Therapies

Use of the liposomes can also be combined with other therapies to treat a given condition. For example, when the methods of the invention are used to treat hypercalcemia, the administration of liposomes can be combined with the administration of a therapeutic agent useful for reducing calcium load in an animal (e.g. pamidronate, furosemide, diphophonates, gallium nitrate, glucocorticoids, zolendranate, etidronate, or calcitonin).

Liposomal Forming Lipids

The liposomes include some liposome forming lipids (e.g. a phosphatidyl choline or sphingomyelin). Typically, the lipids include at least one phosphatidyl choline, which provides the primary packing/entrapment/structural element of the liposome. Typically, the phosphatidyl choline comprises mainly C₁₆ or longer fatty-acid chains. Chain length provides for both liposomal structure, integrity, and stability. Optionally, one of the fatty-acid chains have at least one double bond.

As used herein, the term “phosphatidyl choline” includes Soy PC, Egg PC dielaidoyl phosphatidyl choline (DEPC), dioleoyl phosphatidyl choline (DOPC), distearoyl phosphatidyl choline (DSPC), hydrogenated soybean phosphatidyl choline (HSPC), dipalmitoyl phosphatidyl choline (DPPC), 1-palmitoyl-2-oleo phosphatidyl choline (POPC), dibehenoyl phosphatidyl choline (DBPC), and dimyristoyl phosphatidyl choline (DMPC).

As used herein, the term “Soy-PC” refers to phosphatidyl choline compositions including a variety of mono-, di-, tri-unsaturated, and saturated fatty acids. Typically, Soy-PC includes palmitic acid present in an amount of about 12% to about 33% by weight; stearic acid present in an amount of about 3% to about 8% by weight; oleic acid present in an amount of about 4% to about 22% by weight; linoleic acid present in an amount of about 60% to about 66% by weight; and linolenic acid present in an amount of about 5% to about 8% by weight.

As used herein, the term “Egg-PC” refers to a phosphatidyl choline composition including, but not limited to, a variety of saturated and unsaturated fatty acids. Typically, Egg-PC comprises palmitic acid present in an amount of about 34% by weight; stearic acid present in an amount of about 10% by weight; oleic acid present in an amount of about 31% by weight; and linoleic acid present in an amount of about 18% by weight.

Cholesterol

Cholesterol typically provides stability to the liposome.

The ratio of phosphatidyl choline to cholesterol is from about 0.5:1 to about 4:1 by mole ratio. Preferably, the ratio of phosphatidyl choline to cholesterol is from about 1:1 to about 2:1 by mole ratio. More preferably, the ratio of phosphatidyl choline to cholesterol is about 2:1 by mole ratio.

Preparation of Liposomes

The liposomes comprise a lipid layer of phospholipids and cholesterol. Typically, the ratio of phospholipid to cholesterol is sufficient to form a liposome that will not dissolve or disintegrate once administered to the animal. The phospholipid and cholesterol are dissolved in a suitable solvent or solvent mixture with an appropriate amount of ionophore. After a suitable amount of time, the solvent is removed via vacuum drying or spray drying. The resulting solid material can be stored or used immediately.

Subsequently, the resulting solid material is hydrated in aqueous solution containing, as an example, a calcium sequestering agent, such as EDTA, at appropriate temperatures, resulting in multilamellar vesicles (MLV). The solutions containing MLV are size-reduced via homogenization to form Small Unilameller Vesicles (SUV). A portion of the calcium sequestering agent is encapsulated in the aqueous compartment of SUV during the process. The unencapsulated sequestering agent is removed via suitable methods, such as dialysis, desalting column, or cross filtration. The resulting liposome solution is filtered and ready for use.

Calcium sequestering agent loading in buffer is formulation dependent. Among the relevant factors is the effective phase transition temperature of the lipid components (Tm). FIG. 1, illustrates the lipid transition temperature dependence of calcium loading for various preparations. The loading in buffer may or may not directly relate to loading in vitro (serum, plasma or blood) or in vivo. Serum proteins or other elements may influence (positively or negatively) the function of the transporter channel. The overall liposome performance will result in part from loading in vivo and the biodistribution, pharmacokinetics, and/or pharmacodynamics of the liposome.

Formulations

The lipid-based dispersions of the invention can also be formulated to be administered parenterally. Moreover, the lipid-based dispersions can be formulated for subcutaneous, intramuscular, intravenous, or intraperitoneal administration by infusion or injection. These preparations may also contain a preservative to prevent the growth of microorganisms, buffers, or anti-oxidants in suitable amounts.

Compositions and preparations will typically contain at least 0.1% of the sequestering agent. The percentage of the compositions and preparations may, of course, be varied and may conveniently be between about 2% to about 60% of the weight of a given unit dosage form. The amount of sequestering agent active in such therapeutically useful compositions is such that an effective dosage level will be obtained.

The liposome may also comprise physiologically acceptable salts to maintain isotonicity with animal serum. Any pharmaceutically acceptable salt that achieves isotonicity with animal serum is acceptable, such as NaCl.

The amount of formulation required for use in treatment will vary not only with particular agent but also with the route of administration, the nature of the condition being treated and the age and condition of the animal; the amount required will be ultimately at the discretion of the attendant physician or clinician.

The desired amount of a formulation may conveniently be presented in a single dose or as divided doses administered at appropriate intervals, for example, as two, three, four or more sub-doses per day. The sub-dose itself may be further divided, e.g., into a number of discrete loosely spaced administrations.

Calcium loading was studied in vitro by diluting liposomes in buffer or biological media (e.g. serum) and incubating (e.g. at 37° C.). Over the course of incubation, samples are microfiltered through a small diameter cutoff (<100 kDa) microfiltration device. Filtrates are assayed by high performance liquid chromatography for total calcium concentration. These filtrate concentrations represent the amount of free calcium found in the media. Thus a decrease in filtrate calcium can be attributed to liposome uptake of calcium from the media.

FIG. 1 illustrates the lipid transition temperature dependence of calcium loading in buffer solution for various liposome preparations. Five different formulations of 2:1 phospholipid-cholesterol preparations are shown. The five phospholipids were HSPC (55° C.), DPPC (41° C.), DMPC (23° C.), DEPC (12° C.) and DOPC (−20° C.). All samples were prepared at pH 4.5 with 200 mM EDTA. In the DSPC-cholesterol formulation, two additional preparations were made using ammonium sulfate either entrapped or entrapped and in the exterior buffer.

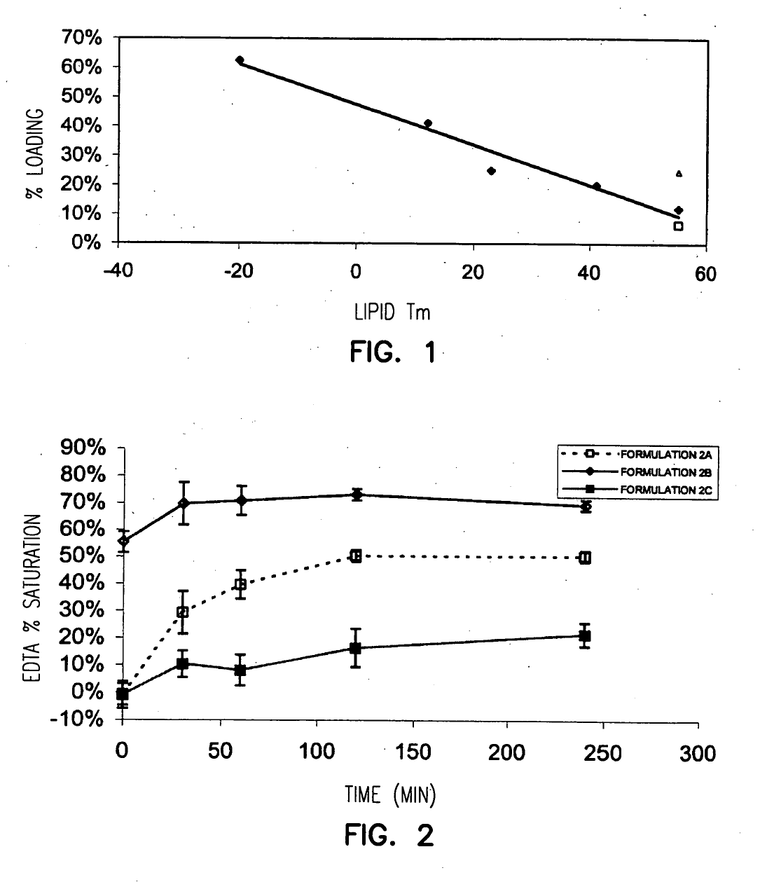

FIG. 2 illustrates calcium loading for three formulations prepared in four different manners. The formulations were prepared using 2:1 HSPC:cholesterol (Formulation 2A); 1:1.25:1.5 HSPC:cholesterol:DOPC (Formulation 2B); and 2:1 DPPC:cholesterol (Formulation 2C), at pH 4.5 with and without 140 mM NaCl or at a pH of 7.5 with and without 140 mM NaCl. Each phospholipid:cholesterol preparation in the 4 different preparations were averaged to obtain the loading curves presented.

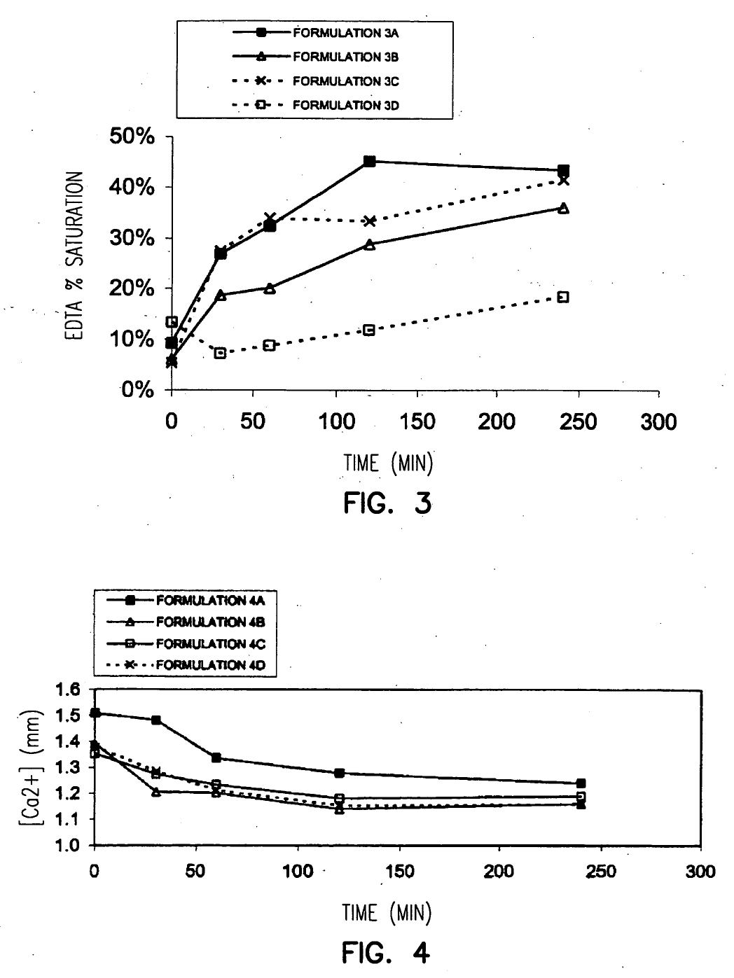

FIG. 3 illustrates the in vitro pH and NaCl dependency of calcium loading efficiency. Four formulations were prepared. A 2:1 DPPC:cholesterol formulation was prepared using 140 mM NaCl and 200 EDTA at a pH of 4.5 (Formulation 3A) and a second at a pH of 7.5 (Formulation 3B). Then a 2:1 DPPC:cholesterol formulation was prepared using 200 EDTA at a pH of 4.5 (Formulation 3C) and a second at a pH of 7.5 (Formulation 3D). Measurements were taken at 0 min, 30 min, 60 min, 120 min, and 240 min. The graph indicates that the formulations prepared at pH of 4.5 contained the highest EDTA % saturation.

FIG. 4 illustrates the calcium loading for four HSPC:cholesterol formulations. In particular, a 2:1 HSPC:cholesterol formulation was prepared using 140 mM NaCl and 200 mM EDTA at a pH of 4.5 (Formulation 4A) and a second at a pH of 7.5 (Formulation 4B). Then a 2:1 HSPC:cholesterol formulation was prepared using 200 mM EDTA at a pH of 4.5 (Formulation 4D) and a second at a pH of 7.5 (Formulation 4D). Measurements were taken at 0 min, 30 min, 60 min, 120 min, and 240 min. The graph indicates that the formulation prepared with 140 mM NaCl and 200 EDTA at pH of 4.5 contained the highest serum saturation.

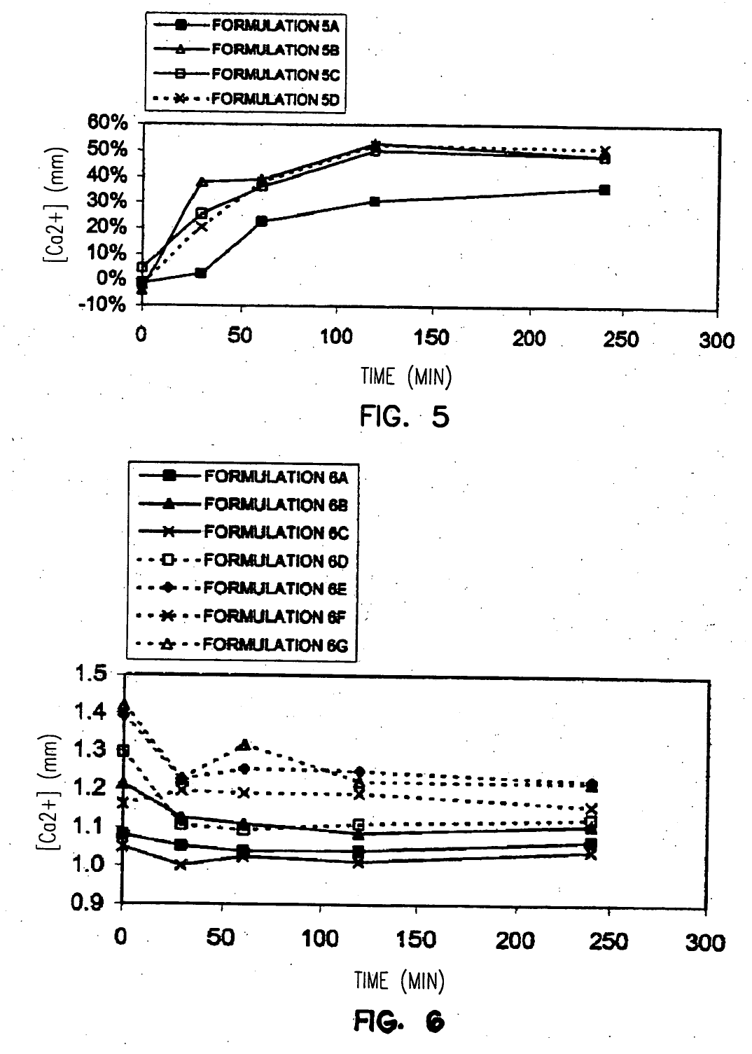

FIG. 5 illustrates the loading efficiency for four HSPC:cholesterol formulations. A 2:1 HSPC:cholesterol formulation was prepared using 140 mM NaCl and 200 mM EDTA at a pH of 4.5 (Formulation 5A) and a second at a pH of 7.5 (Formulation 5B). Then a 2:1 DPPC:cholesterol formulation was prepared using 200 mM EDTA at a pH of 4.5 (Formulation 5D) and a second at a pH of 7.5 (Formulation 5C). Measurements were taken at 0 min, 30 min, 60 min, 120 min, and 240 min. The graph indicates that the formulation prepared without NaCl and with 200 mM EDTA at pH of 4.5 contained the highest calcium ion concentration.

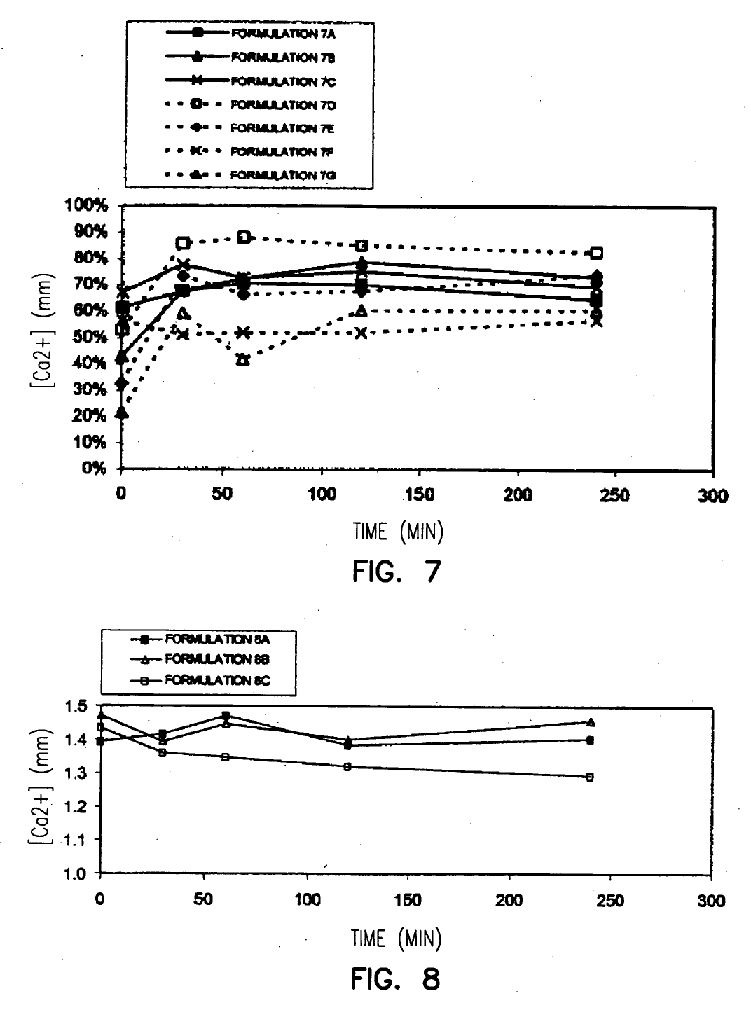

FIG. 6 illustrates the calcium loading for seven HSPC:cholesterol:DOPC formulations. Four 1:1.25:1.5 HSPC:cholesterol:DOPC formulations were prepared: one 140 mM NaCl and 200 mM EDTA at a pH of 4.5 (Formulation 6B), a second at a pH of 7.5 (Formulation 6C), a third without NaCl at a pH of 7.5 (Formulation 6A), and a fourth without NaCl at a pH of 4.5 (Formulation 6D). Three HSPC:cholesterol:DOPC formulations were prepared. One is 1:0.14:0.25 HSPC:cholesterol:DOPC using 200 mM EDTA at a pH of 7.5 (Formulation 6E), a second is 1:0.12:0.05 HSPC:cholesterol:-DOPC using 200 mM EDTA at a pH of 7.5 (Formulation 6F), a third is 1:0.17:0.5 HSPC:cholesterol:DOPC using 200 mM EDTA at a pH of 7.5 (Formulation 6G). Measurements were taken at 0 min, 30 min, 60 min, 120 min, and 240 min FIG. 7 illustrates the loading efficiency for seven HSPC:cholesterol:DOPC formulations. Four 1:1.25:1.5 HSPC:cholesterol:DOPC formulation were prepared: one 140 mM NaCl and 200 mM EDTA at a pH of 4.5 (Formulation 7B), a second at a pH of 7.5 (Formulation 7C), a third without NaCl at a pH of 7.5 (Formulation 7A), and a fourth without NaCl at a pH of 4.5 (Formulation 7D). Three HSPC:cholesterol:DOPC formulations were prepared. One is 1:0.17:0.5 HSPC:cholesterol:DOPC using 200 mM EDTA at a pH of 7.5 (Formulation 7E) a second is 1:0.14:0.25 HSPC:cholesterol:DOPC using 200 mM EDTA at a pH of 7.5 (Formulation 7F) a third is 1:0.12:0.05 HSPC:cholesterol:DOPC using 200 mM EDTA at a pH of 7.5 (Formulation 7G). Measurements were taken at 0 min, 30 min, 60 min, 120 min, and 240 min.

FIG. 8 illustrates the calcium loading for three HSPC:cholesterol:DEPC formulations. Three HSPC:cholesterol:DEPC formulations were prepared. One is 1:0.14:0.25 HSPC:cholesterol:DEPC using 200 mM EDTA at a pH of 7.5 (Formulation 8A) a second is 1:0.12:0.05 HSPC:cholesterol:DEPC using 200 mM EDTA at a pH of 7.5 (Formulation 8B) a third is 1:0.17:0.5 HSPC:cholesterol:DEPC using-200 mM EDTA at a pH of 7.5 (Formulation 8C). Measurements were taken at 0 min, 30 min, 60 min, 120 min, and 240 min.

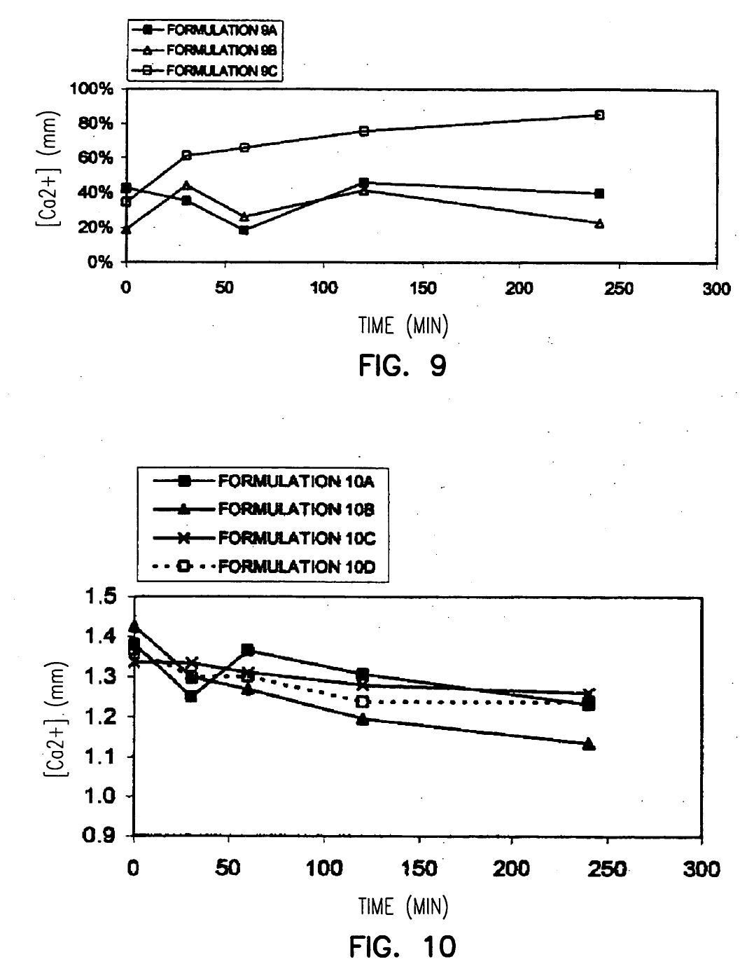

FIG. 9 illustrates the loading efficiency for three HSPC:cholesterol:DEPC formulations. The three HSPC:cholesterol:DEPC formulations were prepared. One is 1:0.17:0.5 HSPC:cholesterol:DEPC using 200 mM EDTA at a pH of 7.5 (Formulation 9A) a second is 1:0.14:0.25 HSPC:cholesterol:DEPC using 200 mM EDTA at a pH of 7.5 (Formulation 9B) a third is 1:0.12:0.05 HSPC:cholesterol:DEPC using 200 mM EDTA at a pH of 7.5 (Formulation 9C). Measurements were taken at 0 min, 30 min, 60 min, 120 min, and 240 min.

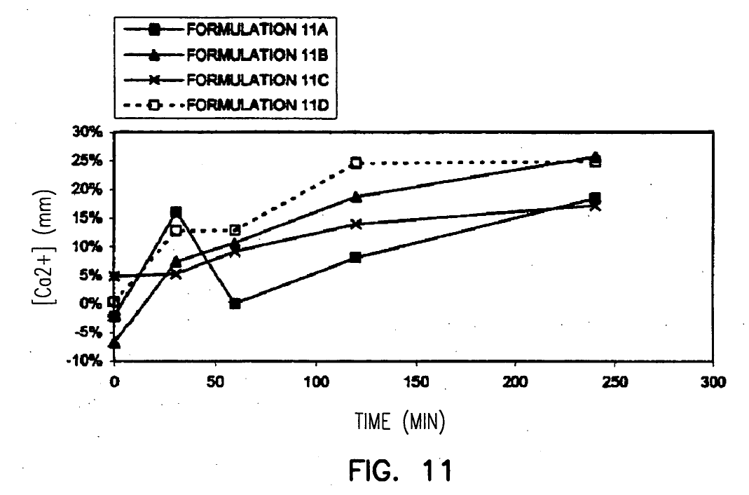

FIG. 10 illustrates the calcium loading for four DPPC:cholesterol formulations. In particular, a 2:1 DPPC:cholesterol formulation was prepared using 140 mM NaCl and 200 EDTA at a pH of 4.5 (Formulation 10D) and a second at a pH of 7.5 (Formulation 10C). Then a 2:1 DPPC:cholesterol formulation was prepared using 200 EDTA at a pH of 4.5 (Formulation 10B) and a second at a pH of 7.5 (Formulation 10A). Measurements were taken at 0 min, 30 min, 60 min, 120 min, and 240 min. The graph indicates that the formulation prepared with 140 mM NaCl and 200 mM EDTA at pH of 7.5 contained the highest calcium ion concentration.

FIG. 11 illustrates the loading efficiency for four DPPC:cholesterol formulations. A 2:1 DPPC:cholesterol formulation was prepared using 140 mM NaCl and 200 mM EDTA at a pH of 4.5 (Formulation 11 D) and a second at a pH of 7.5 (Formulation 11C). Then a 2:1 DPPC:cholesterol formulation was prepared using 200 mM EDTA at a pH of 4.5 (Formulation 11B) and a second at a pH of 7.5 (Formulation 11A). Measurements were taken at 0 min, 30 min, 60 min, 120 min, and 240 min. The graph indicates that the formulation prepared with 140 mM NaCl and 200 mM EDTA at pH of 4.5 contained the highest calcium ion concentration.

The invention is further defined by reference to the following examples describing the preparation of the liposomes and methods of treating hypercalcemia using the liposomes. It will be apparent to those skilled in the art, that many modifications, both to materials and methods, may be practiced without departing from the purpose and interest of this invention.

### EXAMPLES

### Example 1

### General Liposome Preparation

Phospholipids and cholesterol in a ratio of about 2:1, respectively, are dissolved in suitable solvent or solvent mixtures with appropriate amount of ionophore. The solvent is removed subsequently via vacuum drying or spray drying. The resulting solid material can be stored or used immediately.

Subsequently, the resulting solid material is hydrated in aqueous solution containing EDTA at appropriate temperatures, resulting in multilamellar vesicles (MLV). The solutions containing MLV are size-reduced via homogenization to form Small Unilameller Vesicles (SUV). A portion of the EDTA is encapsulated in the aqueous compartment of SUV during the process. The unencapsulated EDTA is removed via suitable methods, such as dialysis, desalting column, or cross filtration. The resulting liposome solution is filtered and ready for use.

All publications, patents, and patent documents are incorporated by reference herein, as though individually incorporated by reference.

What is claimed is:

1. A method for incorporating an entity from an animal into a liposome comprising administering to an animal in need of such treatment liposomes capable of incorporating the entity.

2. A method for incorporating an entity from a biological sample into a liposome comprising contacting the biological sample with one or more liposomes capable of incorporating the entity.

3. The method of claim 1 wherein the entity is an alkali metal, an alkaline earth metal, Fe, Os, Co, Ni, Pd, Cu, Ag, Au, Zn, Al, Cd, Hg, Sn, or Pb, or an ion thereof.

4. The method of claim 1 wherein the entity is calcium or an ion thereof.

5. The method of claim 1 wherein the entity is glucose.

6. The method of claim 1 wherein the liposome comprises a channel or shuttle to facilitate incorporation of the entity into the liposome.

7. The method of claim 1 wherein the pH of the interior of the liposome is less than about 8 prior to administration or contact with the biological sample.

8. The method of claim 1 wherein the entity binds with a sequestering agent inside the liposome to form a complex that is incapable of passing out of the liposome.

9. The method of claim 8 wherein the sequestering agent is EDTA.

10. The method of claim 9 wherein the concentration of EDTA in the liposome is at least about 100 mM prior to administration or contact with the biological sample.

11. The method of claim 1 wherein the entity reacts with a reagent inside the liposome to form a reacted entity.

12. The method of claim 1 wherein the reacted entity is incapable of passing out of the liposome.

13. A method for treating Parkinson's disease in an animal comprising administering an amount of a liposome to the animal that is effective to lower the level of Mn, Fe, Hg, Al, or Cu, or an ion thereof in the animal's brain.

14. A method for treating Alzheimer's disease in an animal comprising administering an amount of a liposome to the animal that is effective to lower the level of Zn, Al, Fe or Cu, or an ion thereof in the animal's brain.

15. A method for treating diabetes in an animal comprising administering an amount of a liposome to the animal that is effective to lower the level of glucose in the animal's serum.

16. A method for removing an entity from the cerebral spinal fluid of an animal in need of such treatment comprising interthecally administering to the animal liposomes capable of incorporating the entity.

17. A method for reducing serum calcium load in an animal in need of such treatment comprising administering an effective calcium reducing amount of one or more liposomes capable of incorporating calcium from the animal.

18. The method of claim 17 wherein the calcium binds with the sequestering agent inside the liposome to form a calcium complex that is incapable of passing out of the liposome.

19. The method of claim 18 wherein the sequestering agent is EDTA

20. The method of claim 19 wherein the concentration of EDTA in the liposome is at least about 100 mM prior to administration.
